(19) United States
(12) Patent Application Publication
(10) Pub. No.: US 2009/0042784 A1
(43) Pub. Date: Feb. 12, 2009

(54) Purification of a Drug Substance of a Factor VII Polypeptide by Removal of DesGla-Factor VII Polypeptide Structures

(76) Inventor: Janus Krarup, Gentofte (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/664,036

(22) PCT Filed: Sep. 29, 2005
(86) PCT No.: PCT/EP2005/054902

§ 371 Date: Feb. 15, 2008

Related U.S. Application Data
(60) Provisional application No. 60/618,756, filed on Oct. 14, 2004.

(30) Foreign Application Priority Data
Sep. 29, 2004 (DK) PA 2004 01478
Sep. 29, 2004 (DK) PA 2004 01480

(51) Int. Cl. A61K 38/37

(52) U.S. Cl. 514/12; 530/383

(57) ABSTRACT

The present invention relates to a purification process for drug substances of a Factor VII polypeptide having an impurity in the form of desGla-Factor VII polypeptide structures. The process utilizes an anion-exchange material and includes washing and/or elution with a buffer of a predetermined pH.

### FIELD OF THE INVENTION

The present invention relates to a purification process for drug substances of a Factor VII polypeptide having an impurity in the form of desGla-Factor VII polypeptide structures. The process utilizes an anion-exchange material and includes washing and/or elution with a buffer of a predetermined pH.

### BACKGROUND OF THE INVENTION

The proteins involved in the clotting cascade, including, e.g., Factor VII, Factor VIII, Factor IX, Factor X, and Protein C, are proving to be useful therapeutic agents to treat a variety of pathological conditions. Accordingly, there is an increasing need for formulations comprising these proteins that are pharmaceutically acceptable and exhibit a uniform and predetermined clinical efficacy.

The overall industrial-scale process for the purification of drug substances of a Factor VII polypeptides may in certain instances suffer from the drawback that the drug substance comprises a considerable amount of corresponding desGla-Factor VII polypeptide structures, i.e. the drug substance is considered to include a considerable amount of an impurity. This is—of course—undesirable and in some instances also unacceptable.

To the present inventors' knowledge, the problem of desGla-Factor VII polypeptide formation and removal in industrial-scale processes has not been fully addressed in the prior art.

### DESCRIPTION OF THE INVENTION

The present inventors have now found that by following a particular anion-exchange procedure wherein the pH is kept low in the most crucial step(s), it is possible to reduce, or virtually eliminate, the presence of desGla-Factor VII polypeptide structures in the drug substance.

The present invention provides a process for the purification of a drug substance of a Factor VII polypeptide, said drug substance comprising at least 3% of desGla-Factor VII polypeptide structures, said process comprising the steps of:

(a) contacting the drug substance with an anion-exchange material under conditions which facilitate binding of a portion of said drug substance to said anion-exchange material;

(b) washing said anion-exchange material with a washing buffer; and

(c) eluting said anion-exchange material with an elution buffer, and collecting a purified drug substance of the Factor VII polypeptide as an eluate;

wherein the loading buffer and/or washing buffer and/or the elution buffer has/have a pH in the range of 2.0-6.9.

The process according to the invention is, thus, particularly relevant for drug substances of Factor VII polypeptides that have a considerable impurity of desGla-Factor VII polypeptide structures, namely at least 3% of desGla-Factor VII polypeptide structures. It should be understood that, in some instances, industrial-scale drug substances of Factor VII polypeptides may include even higher amounts of desGla-Factor VII polypeptide structures, e.g. at least 4%, such as at least 4.5%, or at least 5%, of desGla-Factor VII polypeptide structures, and that the process of the present invention is even more relevant for such drug substances.

When used in conjunction with Factor VII polypeptides, the percentage (%) of desGla-Factor VII polypeptide structures is stated as percentage by weight.

The expression “desGla-Factor VII polypeptide structures” is intended to mean Factor VII polypeptide structures in which the Gla-domain is cleaved from the Factor VII polypeptide molecule in the vicinity of Lys38.

The content of desGla-Factor VII polypeptide structures in a drug substance of a Factor VII polypeptide can be determined by SDS-PAGE as described in Example 1.

Although not limited thereto, the process of the present invention is particularly feasible for “industrial-scale” (or “large-scale”) drug substances of a Factor VII polypeptide. By the term “industrial-scale” is typically meant methods wherein the volume of liquid Factor VII polypeptide compositions is at least 100 L, such as at least 500 L, e.g. at least 1000 L, or at least 5000 L, or where the weight of the compositions is at least 100 kg, such as at least 500 kg, e.g. at least 1000 kg, or at least 5000 kg, or where the weight of the product is at least 1 g (dry matter), such as at least 10 g, e.g. at least 50 g, e.g. 1-1000 g or 1-500 g or 1-100 g.

The expression “drug substance” used herein is intended to mean a solid mass as well as a liquid mass, e.g. a solution or suspension comprising the Factor VII polypeptide. The expression “drug substance” is in particular meant to refer to a “large” volume or mass, i.e. referring to volumes and masses known from large-scale and industrial-scale processes.

The term “Factor VII polypeptide” is defined further below.

Step (a)—Contacting the Drug Substance with an Anion-Exchange Material

In a first step of the process, the drug substance of the Factor VII polypeptide is contacted with an anion-exchange material. The aim is to facilitate binding of a portion of said drug substance of the Factor VII polypeptide to said anion-exchange material.

By the term “portion” in connection with step (a) is meant at least 30% (i.e. 30-100%) of the mass of the Factor VII polypeptide present in the drug substance of the Factor VII polypeptide. It should be understood that it in most instances is desirable to bind far more than 30% of the mass of the Factor VII polypeptides, e.g. at least 50%, or at least 70%, or a predominant portion. By the term “predominant portion” is meant at least 90% of the mass of the Factor VII polypeptide present in the drug substance of the Factor VII polypeptide. Preferably an even higher portion becomes bound to the anion-exchange material, e.g. at least 95% of the mass, or at least 98% of the mass, or at least 99% of the mass, or even substantially all of the mass of the Factor VII polypeptide present in the drug substance of the Factor VII polypeptide.

The drug substance of the Factor VII polypeptide typically originates from an industrial-scale production process, e.g. a cell culture, a cloned animal (e.g. cows, pigs, sheep, goats, and fish) or insect, or the like, in particular from a cell culture.

The anion-exchange material is preferably a strong anion-exchange material, e.g. an anion-exchange material having quaternary ammonium groups. Commercial examples of such materials are DEAE Sepharose, Blue Sepharose, and Q-Sepharose Fast Flow from Amersham Biosciences, and POROS HQ 50 from PerSeptive Biosystems or Tosohaas.

The most common arrangement of the anion-exchange material is in the format of a column. Arrangement in a batch container is of course also possible.

The drug substance of the Factor VII polypeptide is typically obtained directly from a preceding purification step, or from a preceding purification step with subsequent adjustment of pH, ionic strength, chelation of divalent metal ions, etc., whatever necessary.

The pH of the drug substance before and upon application to the anion-exchange material appears to play a relevant role for the formation of desGla-Factor VII polypeptide structures. Thus, it is preferred that the drug substance is in liquid form and has a pH in the range of 2.0-6.9, such as in the range of 3.0-6.5 or 3.5-6.9, upon application to the anion-exchange material. In some interesting embodiments, the drug substance has a pH in the range of 2.0-6.8, such as 3.0-6.8 or 3.5-6.8, or a pH in the range of 2.0-6.7, such as 3.0-6.7 or 3.5-6.7, or a pH in the range of 2.0-6.6, such as 3.0-6.6 or 3.5-6.6, or a pH in the range of 2.0-6.5, such as 3.0-6.5 or 3.5-6.5, or a pH in the range of 2.0-6.4, such as 3.0-6.4 or 3.5-6.4.

Typically, but without limitation, the conductivity is in the range of 5-30 mS/cm, such as 10-20 mS/cm. The temperature of the drug substance is typically, but without limitation, 0-15° C., such as around 2-10° C.

The temperature of the anion-exchange material with the bound Factor VII polypeptide is typically, but without limitation, 0-15° C., such as around 2-10° C., e.g. kept within a specified range by using a cooling jacket and solutions of controlled temperature.

The contacting of the drug substance of the Factor VII polypeptide is typically conducted according to conventional protocols, i.e. the concentration, temperature, ionic strength, etc. of the drug substance may be as usual, and the anion-exchange material may be washed and equilibrated before application as usual.

The load of Factor VII polypeptide is typically in the range of 10-40 g, e.g. 15-30 g, Factor VII polypeptide per litre of matrix (anion-exchange material in wet form), and the drug substance is typically applied at a flow of 3-200 column volumes per hour (CV/h), such as at least 10 CV/h, e.g. at least 20 CV/h or at least 40 CV/h or at least 80 CV/h, e.g. 80-120 CV/h.

Step (b)—Washing Step

After binding of the drug substance of the Factor VII polypeptide to the anion-exchange materials, a washing step (b) is conducted in order to remove a substantial fraction of the desGla-Factor VII polypeptide structures from the anion-exchange material. By this step, the remaining (bound) fraction of the Factor VII polypeptide on the anion-exchange material will have a much lower abundance of desGla-Factor VII polypeptide structures.

This washing step (b) is preferably done with a washing buffer having a pH in the range of 2.0-6.9, such as in the range of 3.0-6.5 or 3.5-6.9. In some interesting embodiments, the washing buffer has a pH in the range of 2.0-6.8, such as 3.0-6.8 or 3.5-6.8, or a pH in the range of 2.0-6.7, such as 3.0-6.7 or 3.5-6.7, or a pH in the range of 2.0-6.6, such as 3.0-6.6 or 3.5-6.6, or a pH in the range of 2.0-6.5, such as 3.0-6.5 or 3.5-6.5, or a pH in the range of 2.0-6.4, such as 3.0-6.4 or 3.5-6.4.

The washing step (b) is typically conducted at a flow of 3-200 column volumes per hour (CV/h), such as at least 10 CV/h, e.g. at least 20 CV/h or at least 40 CV/h or at least 80 CV/h, e.g. 80-120 CV/h.

The washing buffer is typically an aqueous solution comprising a buffering agent, typically a buffering agent comprising at least one component selected from the groups consisting of acids and salts of MES, PIPES, ACES, BES, TES, HEPES, TRIS, histidine, imidazole, glycine, glycylglycine, glycinamide, phosphoric acid, acetic acid (e.g. sodium acetate), lactic acid, glutaric acid, citric acid, tartaric acid, malic acid, maleic acid, and succinic acid. It should be understood that the buffering agent may comprise a mixture of two or more components, wherein the mixture is able to provide a pH value in the specified range. As examples can be mentioned acetic acid and sodium acetate, etc.

The washing buffer may also comprise salts, etc., typically a concentration of anions which are insufficient to perform an elution of the Factor VII polypeptide from the column. At pH 6.0 the washing buffer may have a composition of 100-250 mM NaCl, about 10 mM histidine (buffering agent), pH 6.0.

It should be understood that the washing step (b) may be conducted by using one, two or several different washing buffers, or by the application of a gradient washing buffer.

Step (c)—Elution Step

After the washing step(s) (c), the anion-exchange material is eluted with an elution buffer, and a purified drug substance of the Factor VII polypeptide is collected as an eluate.

A great deal of variability is possible for the elution step (c). If the elution is conducted fairly rapidly, formation of significant amounts of desGla-Factor VII polypeptide structures can be suppressed, even if elution is conducted at a pH above 6.9. However, in order to avoid formation of desGla-Factor VII polypeptide structures, it is preferred that the elution buffer also has a pH in the range of 2.0-6.9, such as in the range of 3.0-6.5 or 3.5-6.9. In some interesting embodiments, the elution buffer has a pH in the range of 2.0-6.8, such as 3.0-6.8 or 3.5-6.8, or a pH in the range of 2.0-6.7, such as 3.0-6.7 or 3.5-6.7, or a pH in the range of 2.0-6.6, such as 3.0-6.6 or 3.5-6.6, or a pH in the range of 2.0-6.5, such as 3.0-6.5 or 3.5-6.5, or a pH in the range of 2.0-6.4, such as 3.0-6.4 or 3.5-6.4.

The elution step (b) is typically conducted at a flow of 3-200 column volumes per hour (CV/h), such as, e.g. at least 10 CV/h or at least 20 CV/H, e.g. 20-60 CV/h.

The type of elution is not particularly critical, thus, it is, e.g., possible to elute with a elution buffer comprising divalent cation(s), conduct a competitive elution utilizing a high concentration of certain anions, or to use a pH gradient, or a combination of the before-mentioned.

In one embodiment, elution is effected by means of an elution buffer comprising one or more divalent cation(s). The concentration of the divalent cation(s) is typically at least 5 mM, e.g. at least 10 mM, or even at least 15 mM.

The divalent cation(s) preferably include(s) at least one divalent cation selected from the group consisting of Ca²⁺, Sr²⁺, Mg²⁺, and Ba²⁺. Such divalent cations have a tendency to bind to the Gla-domain of Factor VII polypeptides, and will thereby facilitate liberation of the drug substance of the Factor VII polypeptide from the anion-exchange material. In one preferred embodiment, the divalent cation(s) include(s) Ca²⁺.

The elution buffer typically has a concentration of the divalent cation(s) of at least 5 mM, such as in the range of 5-100 mM, e.g. in the range of 10-40 mM.

In one variant, the elution buffer is a gradient buffer with respect to the divalent cation(s) (e.g. Ca²⁺), e.g. a gradient buffer wherein the initial concentration of the divalent cation(s) (e.g. Ca²⁺) is in the range of 0-20 mM, and the final concentration of the divalent cation(s) (e.g. Ca²⁺) of the gradient buffer is in the range of 15-100 mM.

In another embodiment, the elution step (c) is conducted by competitive elution of the drug substance with one or more anions, e.g. mono-, di- or trivalent anions. Such anions are typically selected from the group consisting of chloride, acetate, malonate, phosphate, carbonate, sulphate, and nitrate; in particular from chloride (Cl⁻), acetate and malonate.

In one variant, the elution buffer has a concentration of Cl⁻ in the range of 100-800 mM, e.g. 200-500 mM. Alternatively, the elution buffer is a gradient buffer with respect to Cl⁻, e.g. a gradient buffer wherein the initial concentration of Cl⁻ is in the range of 0-300 mM, and the final concentration of Cl⁻ is in the range of 300-1000 mM.

In a further variant, the elution buffer has a concentration of malonate in the range of 100-800 mM, e.g. 200-500 mM. Alternatively, the elution buffer is a gradient buffer with respect to malonate, e.g. a gradient buffer wherein the initial concentration of malonate is in the range of 0-300 mM, and the final concentration of malonate is in the range of 300-1000 mM.

In a still further variant, the elution buffer has a concentration of acetate in the range of 100-1000 mM, e.g. 400-800 mM. Alternatively, the elution buffer is a gradient buffer with respect to acetate, e.g. a gradient buffer wherein the initial concentration of acetate is in the range of 0-400 mM, and the final concentration of acetate is in the range of 400-1000 mM.

In still another embodiment, the elution buffer is a gradient buffer with respect to pH. In one variant hereof, the initial pH of the gradient buffer is in the range of 5.0-6.9, and the final pH of the gradient buffer is in the range of 2.5-4.5.

In some embodiments, at least one of the buffers selected from the group of loading buffer, washing buffer, and elution buffer has a pH in the range of 2.0-6.9. In some particularly preferred embodiments, the washing buffer as well as the elution buffer has a pH in the range of 2.0-6.9. In other embodiments, the loading buffer as well as the washing buffer has a pH in the range of 2.0-6.9. In other embodiments, each of the loading buffer, washing buffer and elution buffer has a pH in the range of 2.0-6.9. In other embodiments, only one of the loading, washing and elution buffers has a pH in the range of 2.0-6.9.

When the elution buffer is a pH gradient, it is possible to combine the washing step (b) and the elution step (c), thus in this instance, a pH gradient with an initial pH of in the range of 4.5-6.9 and a final pH in the range of 2.0-4.5 will yield a useful profile where the forefront is discarded and the remaining fractions are collected.

With respect to the elution step (c) in general, it is preferred that the ionic strength of the elution buffer is in the range of 100-1000 mM, such as 200-800 mM.

The term “purified drug substance” means that the resulting drug substance, i.e. the drug substance collected in step (c), has a lower content of desGla-Factor VII polypeptide structures than the drug substance applied in step (a). The term “purification” refers to the process wherein a purified drug substance can be obtained, i.e. the process of the present invention.

It has been found that the process of the invention very efficiently renders it possible to remove desGla-Factor VII polypeptide structures from drug substances of Factor VII polypeptides, and also renders it possible to suppress the formation of such desGla-Factor VII polypeptide structures. Thus, in preferred embodiments, the purified drug substance of the Factor VII polypeptide collected in step (c) comprises at least 1%-point less desGla-Factor VII polypeptide structures compared to the drug substance in step (a).

More particularly, the purified drug substance of the Factor VII polypeptide comprises at the most 2.5%, such as at the most 2.0%, or at the most 1.5%, of desGla-Factor VII polypeptide structures.

Usually, the anion-exchange material is regenerated for the purpose of subsequent use by a sequence of steps.

### PREFERRED EMBODIMENTS

The present process is particularly useful for obtaining a purified drug substance of a Factor VII polypeptide, and if the conditions for the steps (a)-(c) with respect to pH are selected properly, it is even possible to reduce the formation of desGla-Factor VII polypeptide structures and thereby increase the overall yield of the process.

Thus, a preferred embodiment of the present invention provides a process for the purification of a drug substance of a Factor VII polypeptide, said drug substance comprising at least 4% of desGla-Factor VII polypeptide structures, said process comprising the steps of:

(a) contacting the drug substance with an anion-exchange material under conditions which facilitate binding of a portion of said drug substance to said anion-exchange material, said drug substance being in liquid form and having a pH in the range of 2.0-6.9;

(b) washing said anion-exchange material with a washing buffer having a pH in the range of 2.0-6.9; and

(c) eluting said anion-exchange material with an elution buffer, the elution buffer having a pH in the range of 2.0-6.9 and comprising a divalent cation, and collecting a purified drug substance of the Factor VII polypeptide as an eluate, the collected purified drug substance comprising at the most 2.0% of desGla-Factor VII polypeptide structures.

Factor VII Polypeptide

As used herein, the term “Factor VII polypeptide” encompasses wild-type Factor VII (i.e. a polypeptide having the amino acid sequence disclosed in U.S. Pat. No. 4,784,950), as well as variants, derivatives and conjugates of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. The term “Factor VII polypeptide” also encompasses polypeptides, including variants, in which the Factor VIIa biological activity has been substantially modified or somewhat reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to Tissue Factor (TF) and (ii) catalyze the proteolytic cleavage of Factor IX or Factor X to produce activated Factor IX or X (Factor IXa or Xa, respectively).

The term “improved biological activity” refers to FVII polypeptides with i) substantially the same or increased proteolytic activity compared to recombinant wild type human Factor VIIa or ii) to FVII polypeptides with substantially the same or increased TF binding activity compared to recombinant wild type human Factor VIIa or iii) to FVII polypeptides with substantially the same or increased half life in blood plasma compared to recombinant wild type human Factor VIIa.

For the purposes of the invention, biological activity of Factor VII polypeptides (“Factor VII biological activity”) may be quantified by measuring the ability of a preparation to promote blood clotting, cf. Assay 4 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); (iv) measuring hydrolysis of a synthetic substrate by Factor VIIa and/or a Factor VII-related polypeptide (“In Vitro Hydrolysis Assay”, see Assay 1 below); or (v) measuring generation of thrombin in a TF-independent in vitro system (see Assay 3 below).

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay (Assay 4), proteolysis assay (Assay 2), or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay (Assay 4), proteolysis assay (Assay 2), or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS).

Non-limiting examples of Factor VII variants having increased biological activity compared to wild-type FVIIa include FVII variants as disclosed in WO 01/83725, WO 02/22776, WO 02/077218, WO 03/27147, WO 03/37932; WO 02/38162 (Scripps Research Institute); and FVIIa variants with enhanced activity as disclosed in JP 2001061479 (Chemo-Sero-Therapeutic Res Inst.).

Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993).

Examples of Factor VII polypeptides include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota).

The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

In one preferred embodiment, the Factor VII polypeptide is made by DNA recombinant technology (recombinant Factor VII polypeptide).

In some embodiments, the Factor VII polypeptide is human Factor VIIa (hFVIIa), preferably recombinantly made human Factor VIIa (rhVIIa). In some embodiments the Factor VII polypeptide is a PEGylated Factor VII polypeptide, preferably a PEGylated recombinantly made human Factor VIIa.

In other embodiments, the Factor VII polypeptide is a Factor VII sequence variant.

In some embodiments, the Factor VII polypeptide has a glycosylation different from wild-type human Factor VII.

In various embodiments, e.g. those where the Factor VII polypeptide is a Factor VII-related polypeptide or a Factor VII sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the “In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the “In Vitro Hydrolysis Assay” (see Assay 1 below); in other embodiments, the ratio is at least about 2.0; in further embodiments, the ratio is at least about 4.0.

Use of the Purified Drug Substance of the Factor VII Polypeptide

After collection of the fractions corresponding to the purified drug substance of the Factor VII polypeptide, may be formulated into a solution, which may be dispensed into vials and freeze-dried. As an illustrative example of a final product corresponding to the commercially available, recombinantly-made FVII polypeptide composition NovoSeven® (Novo Nordisk A/S, Denmark), can be mentioned a vial (1.2 mg) containing 1.2 mg recombinant human Factor VIIa, 5.84 mg NaCl, 2.94 mg CaCl₂, 2 H₂O, 2.64 mg GlyGly, 0.14 mg polysorbate 80, and 60.0 mg mannitol. This product is reconstituted to pH 5.5 by 2.0 mL water for injection (WFI) prior to use. When reconstituted, the protein solution is stable for use for 24 hours.

The overall manufacture of recombinant activated Factor VII (rFVIIa) is described by Jurlander, et al. in Seminars in Thrombosis and Hemostasis, Vol. 27, No. 4, 2001.

### EXAMPLES

Assays Suitable for Determining Biological Activity of Factor VII Polypeptides

Factor VII polypeptides useful in accordance with the present invention may be selected by suitable assays that can be performed as simple preliminary in vitro tests. Thus, the present specification discloses a simple test (entitled “In Vitro Hydrolysis Assay”) for the activity of Factor VII polypeptides.

In Vitro Hydrolysis Assay (Assay 1)

Native (wild-type) Factor VIIa and Factor VII polypeptide (both hereinafter referred to as “Factor VIIa”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM HEPES, pH 7.4, containing 0.1 M NaCl, 5 mM CaCl₂ and 1 mg/mL bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used for calculating the ratio between the activities of Factor VII polypeptide and wild-type Factor VIIa:

Ratio=(A405 nm Factor VII polypeptide)/(A405 nm Factor VIIa wild-type).

Based thereon, Factor VII polypeptides with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

The activity of the Factor VII polypeptides may also be measured using a physiological substrate such as Factor X (“In Vitro Proteolysis Assay”), suitably at a concentration of 100-1000 nM, where the Factor Xa generated is measured after the addition of a suitable chromogenic substrate (eg. S-2765). In addition, the activity assay may be run at physiological temperature.

In Vitro Proteolysis Assay (Assay 2)

Native (wild-type) Factor VIIa and Factor VII polypeptide (both hereinafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 μL 50 mM HEPES, pH 7.4, containing 0.1 M NaCl, 5 mM CaCl₂ and 1 mg/mL bovine serum albumin, are incubated for 15 min. Factor X cleavage is then stopped by the addition of 50 μL 50 mM HEPES, pH 7.4, containing 0.1 M NaCl, 20 mM EDTA and 1 mg/mL bovine serum albumin. The amount of Factor Xa generated is measured by the addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used for calculating the ratio between the proteolytic activities of Factor VII polypeptide and wild-type Factor VIIa:

Ratio=(A405 nm Factor VII polypeptide)/(A405 nm Factor VIIa wild-type).

Based thereon, Factor VII polypeptide with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

Thrombin Generation Assay (Assay 3)

The ability of Factor VIIa or Factor VII polypeptides to generate thrombin can also be measured in an assay (Assay 3) comprising all relevant coagulation Factors and inhibitors at physiological concentrations (minus Factor VIII when mimicking hemophilia A conditions) and activated platelets (as described on p. 543 in Monroe et al. (1997) Brit. J. Haematol. 99, 542-547, which is hereby incorporated herein as reference).

One-stage Coagulation Assay (Assay 4)

The biological activity of the Factor VII polypeptides may also be measured using a one-stage coagulation assay (Assay 4). For this purpose, the sample to be tested is diluted in 50 mM PIPES-buffer (pH 7.5), 0.1% BSA and 40 μl is incubated with 40 μl of Factor VII deficient plasma and 80 μl of human recombinant tissue factor containing 10 mM Ca2+ and synthetic phospholipids. Coagulation times are measured and compared to a standard curve using a reference standard in a parallel line assay.

Preparation and Purification of Factor VII Polypeptides

Human purified Factor VIIa suitable for use in the present invention is preferably made by DNA recombinant technology, e.g. as described by Hagen et al., Proc. Natl. Acad. Sci. USA 83: 2412-2416, 1986, or as described in European Patent No. 0 200 421 (ZymoGenetics, Inc.).

Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be completely activated by passing it through an ion-exchange chromatography column, such as Mono Q® (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super-coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell.

In the context of the present invention, the purification comprises at least one anion-exchange chromatography step.

If not completely activated by the process of the invention, Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

The following examples illustrate practice of the processes of the invention. These examples are included for illustrative purposes only and are not intended in any way to limit the scope of the invention claimed.

### Example 1

### Determination of Content of desGla-Factor VII Polypeptide Structures

The content of desGla-Factor VII polypeptide structures relative to the full length Factor VII polypeptide structures is determined by SDS-PAGE. 150 μl of sample is added 50 μl of sample buffer (non reducing, NuPAGE) and boiled for 5 mins. A 10 μl sample is loaded onto a 12% BisTris NuPAGE Gel (Invitrogen). The gel is run at 200 Volts, 120 mA for 55 mins. The gel is stained using coomassie brilliant blue solution, destained and dried. The relative desGla-Factor VII polypeptide content is calculated as the area of the desGla-Factor VII polypeptide band divided by the areas of the Factor VII polypeptide band at approx. 50 kDa and desGla-Factor VII polypeptide band at approx. 45 kDa.

### Example A

Anion exchange chromatography is performed on a column (1 cm inner diameter×10 cm length=7.85 ml column volume(CV)) packed with Pharmacia Q-Sepharose Fast Flow, equilibrated with 5 CV of a solution containing 5 mM EDTA, 10 mM histidine, pH 6.0. The load is 40 CVs of a filtered solution containing 1 mg/ml FVIIa analogue including 12% desGla-Factor VII polypeptide structures, followed by a 5 CV wash using 50 mM NaCl, 10 mM histidine, pH 6.0. The elution is performed using a 10 CV gradient from 25 mM NaCl to 50 mM CaCl₂, 25 mM NaCl, buffered at pH 6.0 by 10 mM histidine. The entire purification is carried out at a flow-rate of 20 CV/h and a temperature of 5° C.

The product peak elutes approximately at 12 mM CaCl₂. The eluate is analysed by SDS-PAGE which will indicate a content of desGla-Factor VII polypeptide structure of less than 2% (below the limit of detection).

### Example B

Anion exchange chromatography is performed on a column (1 cm inner diameter×10 cm length=7.85 ml column volume(CV)) packed with Pharmacia Q-Sepharose Fast Flow, equilibrated with 5 CV of a solution containing 5 mM tri sodium citrate, 10 mM tris, pH 8.0. The load is 40 CVs of a filtered solution containing 1 mg/ml FVIIa analogue including 12% desGla-Factor VII polypeptide structures, followed by a 5 CV wash using 50 mM NaCl, 10 mM tris, and pH 8.0. The elution is performed using a 20 CV gradient from 50 mM NaCl to 750 mM NaCl, buffered at pH 6.0 by 10 mM histidine. The entire purification is carried out at a flow-rate of 10 CV/h and a temperature of 5° C.

The product peak elutes approximately at 350 mM NaCl. The eluate is analysed by SDS-PAGE which will indicate a content of desGla-Factor VII polypeptide structure of less than 2% (below the limit of detection).

### Example C

Anion exchange chromatography is performed on a column (1 cm inner diameter×10 cm length=7.85 ml column volume(CV)) packed with Pharmacia Q-Sepharose Fast Flow, equilibrated with 5 CV of a solution containing 5 mM EDTA, 10 mM histidine, pH 6.0. The load is 40 CVs of a filtered solution containing 1 mg/ml FVIIa analogue including 10% desGla-Factor VII polypeptide structures, followed by a 5 CV wash using 50 mM NaCl, 10 mM histidine, pH 6.0. The elution is performed using a 25 CV gradient from 25 mM NaCl, 25 mM citric acid, 25 mM histidine, pH 6 to 25 mM NaCl, 25 mM citric acid, 25 mM histidine, pH 3. The entire purification is carried out at a flow-rate of 20 CV/h and a temperature of 5° C.

### Example D

Anion exchange chromatography is performed on a column (1 cm inner diameter×10 cm length=7.85 ml column volume(CV)) packed with Pharmacia Q-Sepharose Fast Flow, equilibrated with 5 CV of a solution containing 5 mM citrate, 10 mM tris, pH 8.0. The load is 40 CVs of a filtered solution containing 1 mg/ml FVIIa analogue (including 10% desGla-Factor VII polypeptide structures), followed by a 5 CV wash using 150 mM NaCl, 10 mM histidine, pH 6.0. The elution is performed using a 20 CV gradient from 0 mM NaCl to 750 mM NaCl, buffered at pH 6.0 by 10 mM histidine. The entire purification is carried out at a flow-rate of 10 CV/h and a temperature of 5° C.

### Example E

Anion exchange chromatography was performed on a column (1 cm inner diameter×10 cm length=7.85 ml column volume(CV)) packed with POROS 50 HQ, equilibrated with 5 CV of a solution containing 5 mM EDTA, 10 mM histidine, pH 6.0. The load was 40 CVs of a filtered solution containing 1 mg/ml FVIIa analogue (including 10% desGla-Factor VII polypeptide structures), followed by a 5 CV wash using 175 mM NaCl, 10 mM histidine, pH 6.0. The elution was performed using a 25 CV gradient from 50 mM NaCl to 30 mM CaCl₂, 50 mM NaCl, buffered at pH 6.0 by 10 mM histidine. The entire purification was carried out at a flow-rate of 80 CV/h and a temperature of 5° C.

### Reference Example F

Anion exchange chromatography was performed on a column (1 cm inner diameter×10 cm length=7.85 ml column volume(CV)) packed with Q Sepharose FF, equilibrated with 5 CV of a solution containing 10 mM Glygly, pH 8.6. The load was 20 CVs of a filtered solution containing 1 mg/ml FVIIa (including 10% desGla-Factor VII polypeptide structures), followed by a 5 CV wash using 175 mM NaCl, 10 mM Glygly, pH 8.6. The elution was performed using a 25 CV gradient from 50 mM NaCl to 30 mM CaCl₂, 50 mM NaCl, buffered at pH 8.6 by 10 mM Glygly. The entire purification was carried out at a flow-rate of 40 CV/h and a temperature of 5° C.

SDS-PAGE indicated a desGla-Factor VII polypeptide content of 5-10%. Mass balance by RP-HPLC indicated a step yield of 80%, i.e. a loss of about 20%.

1. A process for the purification of a drug substance of a recombinant Factor VII polypeptide, said drug substance comprising at least 3% of desGla-Factor VII polypeptide structures, said process comprising the steps of:
(a) contacting the drug substance with an anion-exchange material under conditions which facilitate binding of a portion of said drug substance to said anion-exchange material;
(b) washing said anion-exchange material with a washing buffer; and
(c) eluting said anion-exchange material with an elution buffer, and collecting a purified drug substance of the Factor VII polypeptide as an eluate;
wherein the loading buffer and/or washing buffer and/or the elution buffer has/have a pH in the range of 2.0-6.9.

2. The process according to claim 1, wherein the drug substance of the Factor VII polypeptide in step (a) comprises at least 4% of desGla-Factor VII polypeptide structures.

3. The process according to claim 1, wherein the drug substance in step (a) is in liquid form and has a pH in the range of 2.0-6.9.

4. The process according to claim 1, wherein the washing buffer has a pH in the range of 2.0-6.9.

5. The process according to claim 1, wherein the elution buffer has a pH in the range of 2.0-6.9.

6. The process according to claim 1, wherein the washing buffer as well as the elution buffer have a pH in the range of 2.0-6.9.

7. The process according to claim 5, wherein the elution buffer has a pH of at the most 6.5.

8. The process according to claim 1, wherein the elution buffer comprises one or more divalent cation(s) selected from the group consisting of Ca²⁺, Sr²⁺, Mg²⁺, and Ba².

9.-13. (canceled)

14. The process according to claim 1, wherein the elution step (c) is conducted by competitive elution of the drug substance with one or more anions selected from the group consisting of chloride, acetate, malonate, phosphate, carbonate, sulphate, and nitrate.

15.-16. (canceled)

17. The process according to claim 1, wherein the elution buffer is a gradient buffer with respect to Cl⁻.

18-19. (canceled)

20. The process according to claim 1, wherein the elution buffer is a gradient buffer with respect to malonate.

21. The process according to claim 20, wherein the initial concentration of malonate of the gradient buffer is in the range of 0-300 mM, and the final concentration of malonate of the gradient buffer is in the range of 300-1000 mM.

22. The process according to claim 1, wherein the elution buffer has a concentration of acetate in the range of 100-1000 mM.

23. The process according to claim 1, wherein the elution buffer is a gradient buffer with respect to acetate.

24. The process according to claim 23, wherein the initial concentration of acetate of the gradient buffer is in the range of 0-400 mM, and the final concentration of acetate of the gradient buffer is in the range of 400-1000 mM.

25. The process according to claim 1, wherein the elution buffer is a gradient buffer with respect to pH.

26. The process according to claim 25, wherein the initial pH of the gradient buffer is in the range of 5.0-6.9, and the final pH of the gradient buffer is in the range of 2.5-4.5.

27. The process according to claim 1, wherein the ionic strength of the elution buffer is in the range of 100-1000 mM.

28. The process according to claim 1, wherein the purified drug substance of the Factor VII polypeptide collected in step (c) comprises at least 1% less desGla-Factor VII polypeptide structures compared to the drug substance in step (a).

29. The process according to claim 28, wherein the purified drug substance of the Factor VII polypeptide comprises at the most 2.0% of desGla-Factor VII polypeptide structures.

30. A process for the purification of a drug substance of a Factor VII polypeptide, said drug substance comprising at least 4% of desGla-Factor VII polypeptide structures, said process comprising the steps of:
(a) contacting the drug substance with an anion-exchange material under conditions which facilitate binding of a portion of said drug substance to said anion-exchange material, said drug substance being in liquid form and having a pH in the range of 2.0-6.9;
(b) washing said anion-exchange material with a washing buffer having a pH in the range of 2.0-6.9; and
(c) eluting said anion-exchange material with an elution buffer, the elution buffer having a pH in the range of 2.0-6.9 and comprising a divalent cation, and collecting a purified drug substance of the Factor VII polypeptide as an eluate, the collected purified drug substance comprising at the most 2.0% of desGla-Factor VII polypeptide structures.
